(19) United States
(12) Patent Application Publication
(10) Pub. No.: US 2020/0187735 A1
(43) Pub. Date: Jun. 18, 2020

(54) Sanitizing Vacuuming Device

(71) Applicant: Sunil Jadonath, Winter Garden, FL (US)

(72) Inventor: Sunil Jadonath, Winter Garden, FL (US)

(21) Appl. No.: 16/218,548

(22) Filed: Dec. 13, 2018

(51) Int. Cl. A47L 7/00; A61L 2/10

(52) U.S. Cl. CPC A47L 7/0061; A47L 5/28; A61L 2202/14; A61L 2/10

(57) ABSTRACT

A sanitizing vacuuming device for sanitary collection of debris includes a vacuum cleaner that comprises a power module, a suction head, and a receptacle. The suction head is operationally coupled to the power module. An intake port is positioned in the suction head. The vacuum cleaner is configured to vacuum particles from a surface through the intake port and into the receptacle. A plurality of first bulbs is coupled to the suction head proximate to a perimeter of the intake port. The plurality of first bulbs is selectively operationally couplable to the power module, positioning the plurality of first bulbs to emit ultraviolet germicidal radiation. The plurality of first bulbs is configured to disinfect the surface and to irradiate the particles that enter the intake port.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

[symbol]

### (2) Description of Related Art including information disclosed under 37 CFR 1.97 and 1.98

The disclosure and prior art relates to vacuuming devices and more particularly pertains to a new vacuuming device for sanitary collection of debris.

### BRIEF SUMMARY OF THE INVENTION

An embodiment of the disclosure meets the needs presented above by generally comprising a vacuum cleaner that comprises a power module, a suction head, and a receptacle. The suction head is operationally coupled to the power module. An intake port is positioned in the suction head. The vacuum cleaner is configured to vacuum particles from a surface through the intake port and into the receptacle. A plurality of first bulbs is coupled to the suction head proximate to a perimeter of the intake port. The plurality of first bulbs is selectively operationally couplable to the power module, positioning the plurality of first bulbs to emit ultraviolet germicidal radiation. The plurality of first bulbs is configured to disinfect the surface and to irradiate the particles that enter the intake port.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a sanitizing vacuuming device according to an embodiment of the disclosure.

FIG. 2 is a bottom view of an embodiment of the disclosure.

FIG. 3 is a front view of an embodiment of the disclosure.

FIG. 4 is a side view of an embodiment of the disclosure.

FIG. 5 is a cross-sectional view of an embodiment of the disclosure.

### DETAILED DESCRIPTION OF THE INVENTION

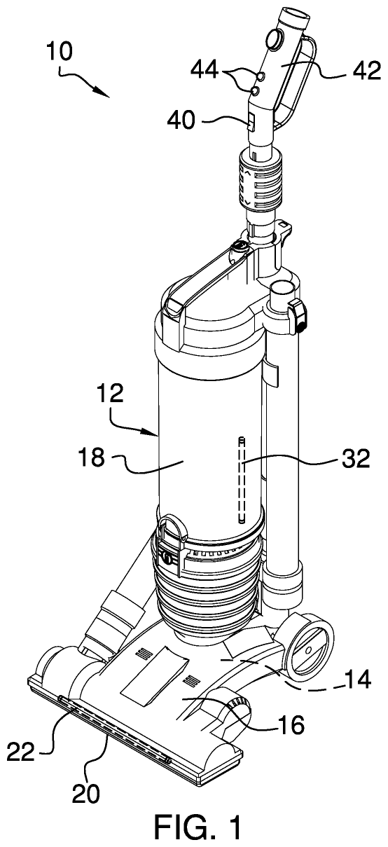

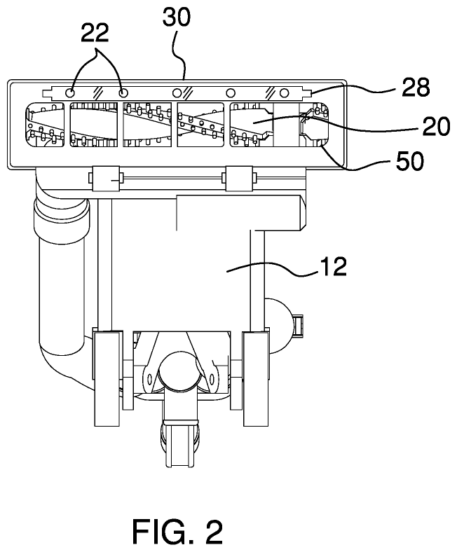

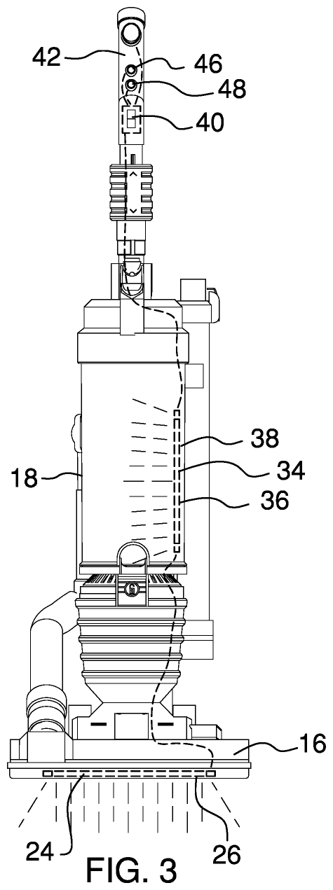

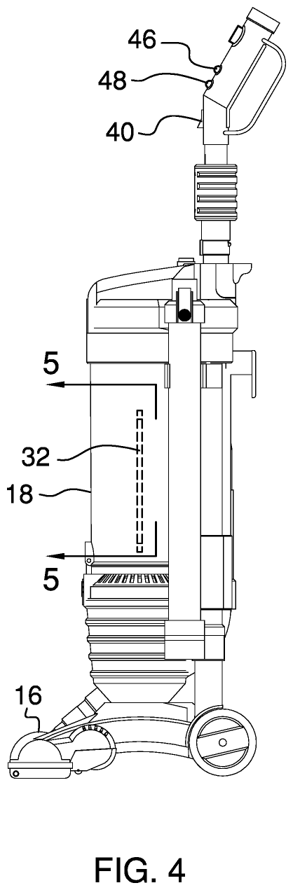

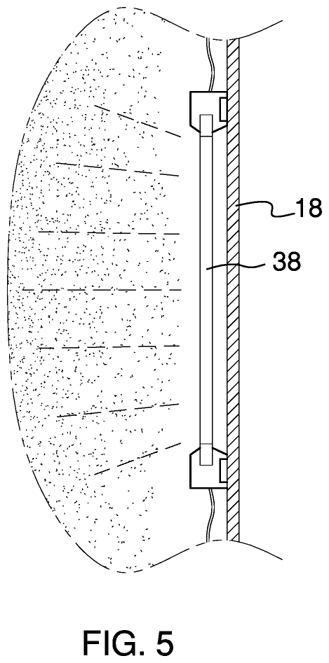

With reference now to the drawings, and in particular to FIGS. 1 through 5 thereof, a new vacuuming device embodying the principles and concepts of an embodiment of the disclosure and generally designated by the reference numeral 10 will be described.

As best illustrated in FIGS. 1 through 5, the sanitizing vacuuming device 10 generally a vacuum cleaner 12 that comprises a power module 14, a suction head 16, and a receptacle 18. The suction head 16 is operationally coupled to the power module 14. The vacuum cleaner 12 may be upright type, canister type, or stick type. An intake port 20 is positioned in the suction head 16. The vacuum cleaner 12 is configured to vacuum particles from a surface through the intake port 20 into the receptacle 18.

A plurality of first bulbs 22 is coupled to the suction head 16 proximate to a perimeter 50 of the intake port 20. The plurality of first bulbs 22 is selectively operationally couplable to the power module 14, positioning the plurality of first bulbs 22 to emit ultraviolet germicidal radiation. The plurality of first bulbs 22 is configured to disinfect the surface and to irradiate the particles that enter the intake port 20.

The plurality of first bulbs 22 comprises at least one of an intake high output germicidal ultraviolet lamp 24 and an intake ultraviolet light emitting diode 26. The plurality of first bulbs 22 comprises an intake strip of bulbs 28 that is positioned between the perimeter of the intake port 20 and a front edge 30 of the suction head 16.

A plurality of second bulbs 32 is coupled to and positioned in the receptacle 18. The plurality of second bulbs 32 is selectively operationally couplable to the power module 14, positioning the plurality of second bulbs 32 is to emit ultraviolet germicidal radiation. The plurality of second bulbs 32 is configured to disinfect the particles in the receptacle 18.

The plurality of second bulbs 32 comprises at least one of a bin high output germicidal ultraviolet lamp 34 and a bin ultraviolet light emitting diode 36. The plurality of second bulbs 32 comprises a bin strip of bulbs 38 that is positioned longitudinally within the receptacle 18.

A switch 40 is coupled to a handle 42 of the vacuum cleaner 12 so that the switch 40 is positioned proximate to a hand of user that is grasping the handle 42. The switch 40 is operationally coupled to the power module 14, the plurality of first bulbs 22, and the plurality of second bulbs 32. The switch 40 is positioned to selectively couple the plurality of first bulbs 22 and the plurality of second bulbs 32 to the power module 14 to illuminate the plurality of first bulbs 22 and the plurality of second bulbs 32.

An indicator 44 is coupled to the handle 42 proximate to the switch 40 and is operationally coupled to the switch 40. The indicator 44 is positioned to indicate coupling of the plurality of first bulbs 22 and the plurality of second bulbs 32 to the power module 14. The indicator 44 comprises a first light 46 and a second light 48. The first light 46 is operationally coupled to the power module 14 when the plurality of first bulbs 22 and the plurality of second bulbs 32 are decoupled from the power module 14 and. The second light 48 is operationally coupled to the power module 14 when the plurality of first bulbs 22 and the plurality of second bulbs 32 are coupled to the power module 14. The first light 46 is red and the second light 48 is green.

In use, the user operates the vacuum cleaner 12 as per prior art vacuum cleaners. When the user desires to disinfect the surface and to irradiate the particles that enter the intake port 20, the switch 40 is used to couple the plurality of first bulbs 22 and the plurality of second bulbs 32 to the power module 14 to illuminate the plurality of first bulbs 22 and the plurality of second bulbs 32 so that ultraviolet germicidal radiation is emitted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sanitizing vacuuming device comprising:
a vacuum cleaner comprising a power module, a suction head, and a receptacle, the suction head being operationally coupled to the power module;
an intake port positioned in the suction head wherein the vacuum cleaner is configured for vacuuming particles from a surface through the intake port into the receptacle; and
a plurality of first bulbs coupled to the suction head proximate to a perimeter of the intake port, the plurality of first bulbs being selectively operationally couplable to the power module such that the plurality of first bulbs is positioned for emitting ultraviolet germicidal radiation wherein the plurality of first bulbs is configured for disinfecting the surface and for irradiating the particles entering the intake port.

2. The device of claim 1, further including the plurality of first bulbs comprising at least one of an intake high output germicidal ultraviolet lamp and an intake ultraviolet light emitting diode.

3. The device of claim 1, further including the plurality of first bulbs comprising an intake strip of bulbs positioned between the perimeter of the intake port and a front edge of the suction head.

4. The device of claim 1, further including a plurality of second bulbs coupled to and positioned in the receptacle, the plurality of second bulbs being selectively operationally couplable to the power module such that the plurality of second bulbs is positioned for emitting ultraviolet germicidal radiation wherein the plurality of second bulbs is configured for disinfecting the particles in the receptacle.

5. The device of claim 4, further including the plurality of second bulbs comprising at least one of a bin high output germicidal ultraviolet lamp and a bin ultraviolet light emitting diode.

6. The device of claim 4, further including the plurality of second bulbs comprising a bin strip of bulbs positioned longitudinally within the receptacle.

7. The device of claim 4, further including a switch coupled to a handle of the vacuum cleaner such that the switch is positioned proximate to a hand of user grasping the handle, the switch being operationally coupled to the power module, the plurality of first bulbs, and the plurality of second bulbs wherein the switch is positioned for selectively coupling the plurality of first bulbs and the plurality of second bulbs to the power module for illuminating the plurality of first bulbs and the plurality of second bulbs.

8. The device of claim 7, further including an indicator coupled to the handle proximate to the switch, the indicator being operationally coupled to the switch wherein the indicator is positioned for indicating coupling of the plurality of first bulbs and the plurality of second bulbs to the power module.

9. The device of claim 8, further including the indicator comprising a first light and a second light wherein the first light is operationally coupled to the power module when the plurality of first bulbs and the plurality of second bulbs are decoupled from the power module and wherein the second light is operationally coupled to the power module when the plurality of first bulbs and the plurality of second bulbs are coupled to the power module.

10. The device of claim 9, further including the first light being red, the second light being green.

11. The device of claim 1, further including the vacuum cleaner being upright type.

12. The device of claim 1, further including the vacuum cleaner being canister type.

13. The device of claim 1, further including the vacuum cleaner being stick type.

14. A sanitizing vacuuming device comprising:
a vacuum cleaner comprising a power module, a suction head, and a receptacle, the suction head being operationally coupled to the power module;
an intake port positioned in the suction head wherein the vacuum cleaner is configured for vacuuming particles from a surface through the intake port into the receptacle;
a plurality of first bulbs coupled to the suction head proximate to a perimeter of the intake port, the plurality of first bulbs being selectively operationally couplable to the power module such that the plurality of first bulbs is positioned for emitting ultraviolet germicidal radiation wherein the plurality of first bulbs is configured for disinfecting the surface and for irradiating the particles entering the intake port, the plurality of first bulbs comprising at least one of an intake high output germicidal ultraviolet lamp and an intake ultraviolet light emitting diode, the plurality of first bulbs comprising an intake strip of bulbs positioned between the perimeter of the intake port and a front edge of the suction head;
a plurality of second bulbs coupled to and positioned in the receptacle, the plurality of second bulbs being selectively operationally couplable to the power module such that the plurality of second bulbs is positioned for emitting ultraviolet germicidal radiation wherein the plurality of second bulbs is configured for disinfecting the particles in the receptacle, the plurality of second bulbs comprising at least one of a bin high output germicidal ultraviolet lamp and a bin ultraviolet light emitting diode, the plurality of second bulbs comprising a bin strip of bulbs positioned longitudinally within the receptacle;
a switch coupled to a handle of the vacuum cleaner such that the switch is positioned proximate to a hand of user grasping the handle, the switch being operationally coupled to the power module, the plurality of first bulbs, and the plurality of second bulbs wherein the switch is positioned for selectively coupling the plurality of first bulbs and the plurality of second bulbs to the power module for illuminating the plurality of first bulbs and the plurality of second bulbs; and
an indicator coupled to the handle proximate to the switch, the indicator being operationally coupled to the switch wherein the indicator is positioned for indicating coupling of the plurality of first bulbs and the plurality of second bulbs to the power module, the indicator comprising a first light and a second light wherein the first light is operationally coupled to the power module when the plurality of first bulbs and the plurality of second bulbs are decoupled from the power module and wherein the second light is operationally coupled to the power module when the plurality of first bulbs and the plurality of second bulbs are coupled to the power module, the first light being red, the second light being green.

15. The device of claim 14, further including the vacuum cleaner being upright type.

16. The device of claim 14, further including the vacuum cleaner being canister type.

17. The device of claim 14, further including the vacuum cleaner being stick type.
